(19) United States
(12) Patent Application Publication
(10) Pub. No.: US 2008/0015672 A1
(43) Pub. Date: Jan. 17, 2008

(54) GRAFT

(76) Inventor: Oswald S. Binford, Corsicana, TX (US)

Correspondence Address: MICHAEL A. O'NEIL, P.C., 5949 SHERRY LANE, SUITE 820, DALLAS, TX, 75225, US

(21) Appl. No.: 11/560,063

(22) Filed: Nov. 15, 2006

Related U.S. Application Data
(63) Continuation-in-part of application No. 11/457,044, filed on Jul. 12, 2006.

(51) Int. Cl. A61F 2/06

(52) U.S. Cl. 623/1.3; 623/1.37

(57) ABSTRACT

A graft comprises an inner interior surface defined by a relatively large radius and an outer interior surface defined by a relatively small radius for maintaining laminar flow of blood passing through the graft and thereby substantially reducing cellular proliferation and blood clotting.

### CROSS-REFERENCE TO RELATED APPLICATION

This application is a continuation-in-part application of application Ser. No. 11/457,044 filed Jul. 12, 2006, currently pending, the entire contents of which are incorporated herein by reference.

### TECHNICAL FIELD

This invention relates generally to the construction of grafts, and more particularly to an improved graft construction which substantially eliminates cellular proliferation and clotting of blood flowing therethrough.

### BACKGROUND AND SUMMARY OF THE INVENTION

In medicine an anastomosis is a point of surgical connection between two tubular structures. Anastomosis commonly refers to a connection made between two blood vessels. Anastomosis also defines the connection made between a blood vessel and a natural or synthetic graft.

FIG. 1 illustrates a graft G connected between an artery and a vein of a patient P. Other types and kinds of graft placements are well known to those skilled in the art. Regardless of the kind of graft used or where the graft is placed, the function of the graft G is to facilitate withdrawal of blood from the patient P and return of blood to the patient P. It may be desirable to remove and return blood from a patient for any number of reasons, such as for hemodialysis. Those skilled in the art will readily understand the multiple and various reasons for removing blood from, and returning blood to, a patient.

Heretofore conventional grafts have been circular in cross section. Conventional grafts tend to clog with a proliferation of cells and coagulated blood. When this occurs the graft must be surgically declotted or a new graft must be installed at a different location. Graft declotting and replacement are surgical procedures, meaning that the patient must undergo repeated surgeries simply to assure a flow of blood through the graft adequate to facilitate the specific procedure being performed.

When blood flows through a conventional graft the portion thereof flowing through the outside portion of the graft flows at a different rate as compared with the flow of blood through the inside portion of the graft thereby resulting in turbulence. It is accepted by the medical community that turbulence within the graft, as documented by doppler ultrasound, predisposes the graft to failure.

It is theorized that the turbulence within the graft traumatizes the inner wall of the blood vessel at the vessel-graft junction, commonly referred to as the anastamosis. The inner wall of the blood vessel is composed of endothelial cells. In response to this trauma, the endothelial cells proliferate into the lumen of the graft. Proliferation of the endothelium narrows the lumen in the vicinity of the anastamosis thereby increasing the turbulence within the graft and decreasing the blood flow rate within the graft. The increased turbulence results in additional endothelial trauma and subsequent endothelial proliferation. This cumulative process continues until the diminished blood flow within the graft renders the graft unsuitable for use. Without surgical intervention, a blood clot forms throughout the graft due to stagnant blood flow and the patient must have a new graft installed.

The present invention comprises a graft which substantially reduces turbulence in blood flowing therethrough. Because turbulence is substantially eliminated, stimulation for endothelial proliferation within the graft is markedly reduced and the tendency of blood flowing through the graft of the present invention to clot is markedly reduced. This in turn substantially extends the useful life of the graft which in turn results in a significant reduction in the number of surgeries that the patient must endure during treatment.

### BRIEF DESCRIPTION OF THE DRAWINGS

A more complete understanding of the present invention may be had by reference to the following Detailed Description when taken in connection with the accompanying Drawings, wherein:

FIG. 1 is a diagrammatic illustration of a human arm having a graft installed thereon;

FIG. 2 is a perspective view illustrating one embodiment of the graft of the present invention;

FIG. 3 is a sectional view taken along the line 3-3 in FIG. 2 in the direction of the arrows;

FIG. 4 is a perspective view illustrating an anastomosis utilizing a conventional graft wherein blood is flowing from the graft into a blood vessel;

FIG. 4A is a sectional view taken along the line 4A-4A in FIG. 4 in the direction of the arrows;

FIG. 5 is a perspective view illustrating an anastomosis utilizing the graft of the present invention wherein blood is flowing from the graft into a blood vessel;

FIG. 5A is a sectional view taken along line 5A-5A in FIG. 5 in the direction of the arrows;

FIG. 6 is perspective view illustrating an anastomosis utilizing a conventional graft wherein blood is flowing from a blood vessel into the graft;

FIG. 6A is a sectional view taken along line 6A-6A in FIG. 6 in the direction of the arrows;

FIG. 7 is a perspective view illustrating an anastomosis utilizing the graft of the present invention wherein blood is flowing from a blood vessel into the graft; and

FIG. 7A is a sectional view taken along the line 7A-7A in FIG. 7 in the direction of the arrows.

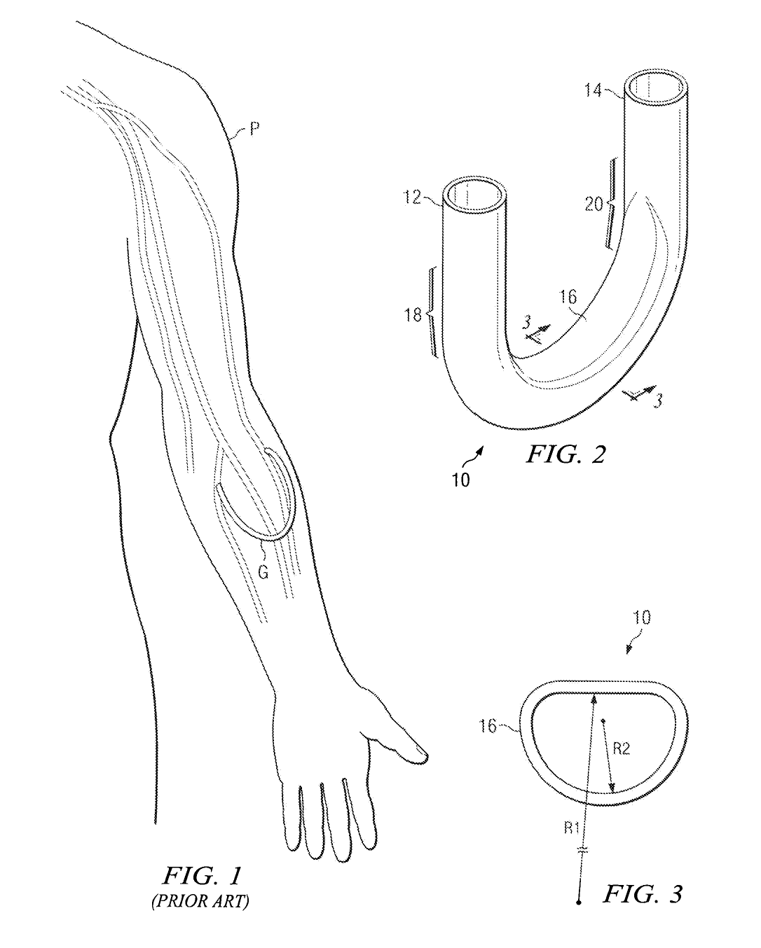

### DETAILED DESCRIPTION

An embodiment of the graft of the present invention is illustrated in FIGS. 2 and 3. A graft 10 comprising the invention has opposite ends 12 and 14 which are round in cross section. Between the ends 12 and 14 the graft 10 comprises a curved section 16 having the generally D-shaped cross sectional configuration illustrated in FIG. 3. The round cross sections comprising the ends 12 and 14 of the graft 10 transition to the generally D-shaped cross sectional configuration illustrated in FIG. 3 in transition zones 18 and 20.

Referring specifically to FIG. 3, the radius R1 defining the inside surface of the graft 10 in the curved section 16 is relatively large as compared with the radius R2 of the outside surface of the graft 10 in the curved section 16. The generally D-shaped cross section of the graft 10 in the curved section 16 thereof minimizes the differential in velocities as between the inner interior surface and the outer interior surface of the graft 10 in the curved section 16 thereby minimizing sheer forces and maintaining laminar flow.

By maintaining laminar flow in blood flowing through the curved section 16 of the graft 10 cellular proliferation and coagulation of the blood is substantially reduced. Reduction in cellular proliferation and coagulation substantially extends the useful life of the graft 10. This in turn substantially reduces the number of surgeries that will be required during treatment of a patient.

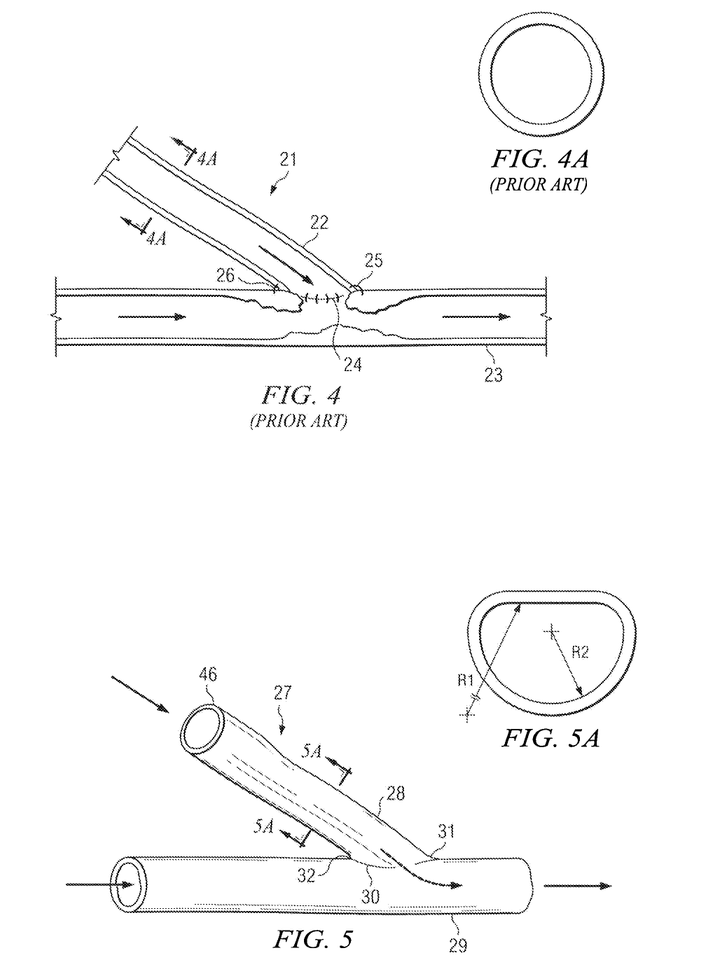

FIG. 4 illustrates an anastomosis 21 wherein a conventional graft 22 is surgically connected to a blood vessel 23 at a vessel-graft junction 24. The arrows represent the direction of blood flow through the graft 22 and the blood vessel 23. In FIG. 4, blood is flowing through the graft 22 into the blood vessel 23.

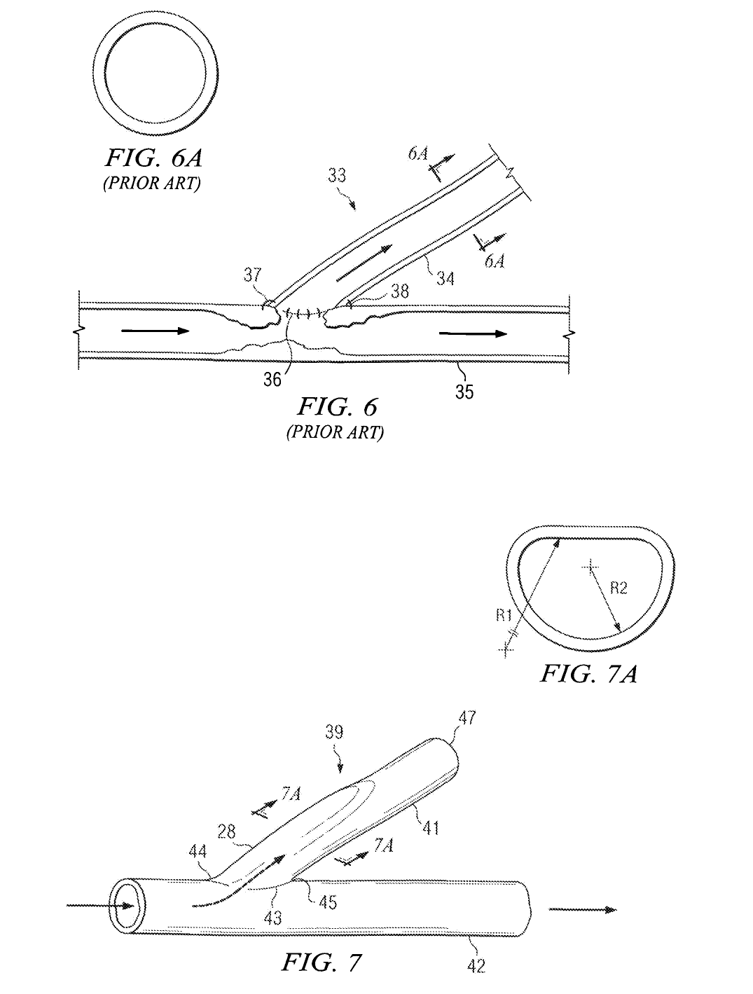

Likewise, referring to FIG. 6, an anastomosis 33 is shown, wherein a conventional graft 34 is surgically connected to a blood vessel 35 at a vessel-graft junction 36. The arrows represent the direction of blood flow through the graft 34 and the blood vessel 35. In FIG. 6, blood is flowing through the blood vessel 35 into the graft

Referring generally to FIGS. 4A and 6A in reference to FIGS. 4 and 6, the grafts 22 and 34 are circular in cross section. Given this circular cross sectional configuration, the grafts 22 and 34 will tend to clog with a proliferation of cells and coagulated blood. This is because when blood flows through the graft 22 into the blood vessel 23 at the vessel-graft junction 24, or through the blood vessel 35 into the graft 34 at the vessel-graft junction 36, the blood flowing through portions 25 and 37 of the vessel-graft junctions 24 and 36 flows at a different rate as compared with the flow of blood through portions 26 and 38 of the vessel-graft junctions 24 and 36, thereby resulting in turbulence.

It is accepted by the medical community that turbulence within a graft as documented by doppler ultrasound, predisposes a graft to failure. It is theorized that the turbulence within a graft traumatizes the inner wall of the blood vessel at the vessel-graft junction.

The inner wall of the blood vessel is composed of endothelial cells. In response to this trauma, the endothelial cells proliferate into the lumen of the graft. Proliferation of the endothelium narrows the lumen in the vicinity of the vessel-graft junction thereby increasing the turbulence within the graft and decreasing the blood flow rate within the graft. The increased turbulence results in additional endothelial trauma and subsequent endothelial proliferation. This cumulative process continues until the diminished blood flow within the graft renders the graft unsuitable for use. When this occurs the graft must be surgically declotted or a new graft must be installed at a different location. Graft declotting and replacement are surgical procedures meaning that a patient must undergo repeated surgeries simply to assure the flow of blood through a graft adequate to facilitate the specific procedure being performed. Without surgical intervention, a blood clot forms throughout the graft due to stagnant blood flow and the patient must have a new graft installed.

Referring now to FIG. 5, an anastomosis 27 is shown. A graft 28 of the present invention is surgically connected to a blood vessel 29 at a vessel-graft junction 30. The arrows represent the direction of blood flow through the graft 28 and the blood vessel 29. In FIG. 5, blood is flowing through the graft 28 into the blood vessel 29.

Likewise, referring to FIG. 7, an anastomosis 39 is shown, wherein a graft 41 of the present invention is surgically connected to a blood vessel 42 at a vessel-graft junction 43. The arrows represent the direction of blood flow through the graft 41 and the blood vessel 42. In FIG. 7, blood is flowing through the blood vessel 42 into the graft 41.

The grafts 28 and 41 comprising the invention have ends 46 and 47 respectively, which are round in cross section. Between the ends 46 and 47 the grafts 28 and 41 have the D-shaped cross sectional configuration illustrated in FIGS. 5A and 7A. The round cross sections comprising the ends 46 and 47 of the grafts 28 and 41 transition to the D-shaped cross sectional configuration illustrated in FIGS. 5A and 7A via transition zones.

Referring to FIGS. 5A and 7A in reference to FIGS. 5 and 7, the radii R1 defining the inner interior surfaces of the grafts 28 and 41 in the D-shaped cross section are relatively large as compared with the radii R2 of the outer interior surfaces of the grafts 28 and 41 in the D-shaped cross section. The generally D-shaped sides of grafts 28 and 41 are oriented so as to coincide with portions 32 and 45 of the vessel-graft junctions 30 and 43 respectively. The generally D-shaped cross sections of the grafts 28 and 41 minimize the differential in velocities at portions 32 and 45 of the vessel-graft junctions 30 and 43 as between portions 31 and 44 of the vessel-graft junctions 30 and 43, thereby minimizing sheer forces and maintaining laminar flow.

By maintaining laminar flow in blood flowing through the grafts 28 and 41 cellular proliferation and coagulation of the blood is substantially reduced. Reduction in cellular proliferation and coagulation substantially extends the useful life of the grafts 28 and 41. This in turn substantially reduces the number of surgeries that will be required during treatment of the patient.

Referring to FIGS. 5 and 7, the angular placement of the grafts 28 and 41 at the vessel-graft junctions 30 and 43 can range from 0 degrees to 180 degrees depending on the specific circumstances. Therefore, it will be necessary to optimize the exact shape of the grafts 28 and 41 at the vessel-graft junctions 30 and 43, according to the parameters of the specific application, the generally D-shaped configuration of the grafts 28 and 41 being maintained. The graft of the present invention can be made of either natural materials, synthetic materials, or a combination thereof.

Although preferred embodiments of the invention have been illustrated in the accompanying Drawings and described in the foregoing Detailed Description, it will be understood that the invention is not limited to the embodiments disclosed, but is capable of numerous rearrangements, modifications, and substitutions of parts and elements without departing from the spirit of the invention.

1. For use in a graft, the improvement comprising:
the graft having an inner interior surface comprising a relatively large radius and an outer interior surface comprising a relatively small radius;
the differential between the radius of the inner interior surface and the radius of the outer interior surface facilitating laminar flow of blood through the graft thereby minimizing cellular proliferation and blood coagulation.

2. The improvement of claim 1 wherein the graft is generally D-shaped.

3. For use in a graft, the improvement comprising:
the graft having a generally D-shaped cross sectional configuration;
the generally D-shaped cross sectional configuration facilitating laminar flow of blood through the graft thereby minimizing cellular proliferation and blood coagulation.

4. A graft, comprising:
an end having a relatively small cross sectional configuration; and
an opposite end having a relatively large cross sectional configuration.

5. The graft of claim 4, wherein the end is round in cross section.

6. The graft of claim 4, wherein the opposite end is generally D-shaped.

7. The graft of claim 4, wherein the round end transitions to the generally D-shaped opposite end via a transition zone.

8. A graft, comprising:
an end which is round in cross section
an opposite end which is generally D-shaped in cross section; and
a transition zone situated between the round end and the generally D-shaped opposite end, wherein the round cross section of the end transitions to the generally D-shaped cross section of the opposite end.

9. A method of facilitating a laminar flow of blood at a vessel-graft junction of an anastomosis between a blood vessel and a graft thereby minimizing cellular proliferation and blood coagulation at the anastomosis comprising the steps of:
predetermining flow parameters at a desired anastomosis between a blood vessel and a graft;
optimizing the graft's shape according to the predetermined flow parameters and specific graft application, the graft having a generally D-shaped cross sectional configuration;
adjusting angular placement of the graft at the anastomosis in accordance with the predetermined flow parameters and in accordance the graft's shape and specific graft application; and
surgically connecting the graft to a blood vessel.

10. The method of claim 5 wherein the blood vessel is a vein.

11. The method of claim 5 wherein the blood vessel is an artery.

12. The method of claim 5 wherein the graft is natural.

13. The method of claim 5 wherein the graft is synthetic.
